(19) United States
(12) Patent Application Publication
(10) Pub. No.: US 2006/0155142 A1
(43) Pub. Date: Jul. 13, 2006

(54) Low pressure process for the preparation of methanedisulfonic acid alkali metal salts

(75) Inventors: Christian Werner, Hannover (DE); Andreas Kanschik-Conradsen, Garbsen (DE); Michael Hoffmeister, Hannover (DE); Harald Heuduk, Wunstorf (DE); Thomas Scholten, Barsinghausen (DE)

Correspondence Address: COLLEEN D. SZUCH;Honeywell International Inc. - Patent Department, 101 Columbia Road, Morristown, NJ, 07962, US

(73) Assignee: Honeywell International Inc.

(21) Appl. No.: 11/033,580

(22) Filed: Jan. 12, 2005

(51) Int. Cl. C07C 309/00

(52) U.S. Cl. 562/80

(57) ABSTRACT

The invention provides a process for producing alkylpolysulfonic acid alkali metal salts such as methanedisulfonic acid alkali metal salts. The process involves heating an aqueous solution of about two molar equivalents of an alkali metal sulfite to a temperature of from about 70° C. to about 90° C.; adding about one molar equivalent of a dihaloalkane to the aqueous alkali metal sulfite solution at a temperature of about 70-90° C.; and then separating the resulting alkyldisulfonic acid alkali metal salt from the reaction solution. The dihaloalkane is added at a pressure of about 1.1 atmospheres or less.

### BACKGROUND OF THE INVENTION

The present invention relates to a process for producing alkylpolysulfonic acids and their alkali metal salts such as methanedisulfonic acid alkali metal salts. More particularly the invention pertains to a process for producing methanedisulfonic acid potassium salt. Such salts find use in aqueous electrolytic plating solutions.

It is known from WO 91/06693 that alkylpolysulfonic acid alkali metal salts find use for electroplating chromium using lead anodes without suffering the excessive anode-corrosion characteristics. Chromium is electrodeposited from a bath containing chromic acid, sulfate and an alkylpolysulfonic acid alkali metal salts containing from one to about three carbon atoms. U.S. Pat. No. 4,588,481, discloses chromium electroplating processes which use baths containing alkylsulfonic acids having a certain ratio of sulfur to carbon, but free of carboxylic acids to produce hard, adherent chromium deposits produced at elevated temperatures and high efficiencies without cathodic low-current-density etching. GB 2153387 teaches electroplating chromium using Group VI salts of methane disulfonic acids.

GB 1,128,860 teaches a method for the production of methanedisulfonic acid salts such as its potassium salt, however, the process requires disadvantageously high temperatures and high pressures, i.e. temperatures of 90° C. to 160° C. at pressures of 4-20 atmospheres.

It has now been found that high yields of alkyldisulfonic acid alkali metal salt can be obtained at lower temperatures and pressures. This addition may be conducted at about atmospheric pressure.

### DESCRIPTION OF THE INVENTION

The invention provides a process for producing an alkyldisulfonic acid alkali metal salt which comprises heating an aqueous solution of about two molar equivalents of an alkali metal sulfite to a temperature of from about 60° C. to about 90° C.; adding about one molar equivalent of a dihaloalkane to the aqueous alkali metal sulfite solution at a temperature of about 60-90° C.; and then separating the resulting alkyldisulfonic acid alkali metal salt from the reaction solution.

The invention also provides a process for producing methanedisulfonic acid potassium salt which comprises heating an aqueous solution of about two molar equivalents of potassium sulfite to a temperature of from about 60° C. to about 90° C.; adding about one molar equivalent of dibromomethane to the aqueous potassium sulfite solution at a temperature of about 60° C. to about 90° C.; and then separating the resulting methanedisulfonic acid potassium salt from the reaction solution.

The invention also provides a process for producing an alkyldisulfonic acid which comprises heating an aqueous solution of about two molar equivalents of an alkali metal sulfite to a temperature of from about 60° C. to about 90° C.; adding about one molar equivalent of a dihaloalkane to the aqueous alkali metal sulfite solution at a temperature of about 60° C. to about 90° C.; separating the resulting alkyldisulfonic acid alkali metal salt from the reaction solution; and then reacting the alkyldisulfonic acid alkali metal salt with an ion exchange polymer.

The first step in the process of the invention forms an aqueous solution of about one molar equivalent of an alkali metal sulfite. One preferred alkali metal sulfite is potassium sulfite. Another useful alkali metal sulfite is sodium sulfite. The aqueous alkali metal sulfite solution is brought to a temperature in the range of from about 60° C. to about 90° C., preferably from about 60° C. to about 80° C.; and more preferably from about 65° C. to about 75° C. About one molar equivalent of a dihaloalkane is slowly added to this aqueous alkali metal sulfite solution. Dihaloalkanes include dibromomethane, diiodomethane, bromochloromethane, bromoiodomethane or combinations thereof. This addition is conducted at a temperature of from about 60° C. to about 90° C., preferably from about 60° C. to about 80° C.; and more preferably from about 65° C. to about 75° C. This addition is conducted for from about 2 hours to about 48 hours, preferably from about 8 hours to about 20 hours; and more preferably from about 12 hours to about 16 hours. The pressure during this addition is preferably about 1.1 atmospheres or less, preferably from about 1.01 atmospheres or less, and more preferably from about 1.005 atmospheres to about 1.000 atmospheres. Preferably this addition is conducted at about atmospheric pressure. Also during this addition, an optional catalyst made be added. Suitable catalysts non-exclusively include an alkali metal iodide, a tetraalkyl ammonium salt, and combinations thereof. Preferred alkali metal iodides include potassium iodide, sodium iodide or lithium iodide and combinations thereof. Preferred tetraalkyl ammonium salts non-exclusively include tetrabutylammonium bromide, methyltributylammonium bromide, methyltributylammonium chloride. The catalyst may be present in an amount of from about 0.01 weight percent to about 10 weight percent based on the weight of the reaction mixture. Preferably the catalyst may be present in an amount of from about 0.1 weight percent to about 1 weight percent, and more preferably from about 0.4 weight percent to about 0.7 weight percent. The reaction mixture remains a homogenous solution rather than a suspension until the alkyldisulfonic acid alkali metal salt is allowed to precipitate out.

Thereafter the produced alkyldisulfonic acid alkali metal salt is separated from the reaction solution such as by washing, precipitation, crystallization, filtration and drying. Washing may be done by one or more contacts with one or more applications and removals of water. Precipitation may be conducted by cooling. Crystallization may be conducted by evaporation of the solvent and cooling. Filtration may be conducted by sucking filtration or centrifugation. Drying may be conducted by heating at from about 80° C. to about 110° C. at atmospheric pressure or under reduced pressure.

Preferably the resulting alkyldisulfonic acid alkali metal salt comprises about 50 ppm or less of bromides, preferably about 25 ppm or less of bromides, and more preferably about 10 ppm or less of bromides. Preferably the resulting alkyldisulfonic acid alkali metal salt comprises about 100 ppm or less of sulfates, preferably about 50 ppm or less of sulfates. The process usually produces a yield of alkyldisulfonic acid alkali metal salt of about 80% or more based on the amount of amount of dihalomethane.

For the preparation of an alkyldisulfonic acid, the alkyldisulfonic acid alkali metal salt is then reacted with an ion exchange polymer. Suitable ion exchange polymers include strong acid ion exchange resins such as sulfonic acid group containing polymers, for example styrene-divinylbenzene or phenol-formaldehyde polymers. Commercially availably sulfonic acid group containing strong acid ion exchange polymers include Permutit RSP-100 commercially available from USFilter PWS Inc. of Colorado Springs, Colo.; Amberlite IR-120, commercially available from Rohm and Haas of Philadelphia, Pa.: and Dowex 50 W-X8 commercially available from The DOW Chemical Company of Midland, Mich. The ion exchanger polymer is placed in a column and a solution of the alkyldisulfonic acid alkali metal salt is transferred drop vise into the column over several hours until the alkali metal is replaced by H+. This is usually conducted at a temperature in the range of from about 20° C. to about 90° C., or more usually from about 20° C. to about 50° C. Water and the corresponding halogen acid can be removed from the alkyldisulfonic acid by distillation at a pressure below 100 mbar within a temperature range from 50 to 130° C.

The following non-limiting examples serve to illustrate the invention.

### EXAMPLE 1

Preparation of K₂[CH₂(SO₃)₂]

Example 1:

Raw Materials:

| | |
|---|---|
| CH₂Br₂: | 140 g/0.8 mol |
| K₂SO₃ (45% in water) | 600 g/1.7 mol |
| [(C₄H₉)₄N]Br: | 6 g |
| KI: | 0.8 g |

A mixture of 600 g K₂SO₃-solution (45% in water), 6 g of [(C₄H₉)₄N]Br and 0.8 g KI in 400 ml of water, was heated to 70° C. Within a period of 16 hours, 140 g of CH₂Br₂ were transferred under the surface of the reaction mixture. During the addition of CH₂Br₂, the pressure in the reactor was determined with 1007-1008 mbar. Afterwards the reaction mixture was heated up to 90° C. After a total reaction time of 24 h, K₂[CH₂(SO₃)₂] started to precipitate. After 72 hours at 90° C., complete conversion of CH₂Br₂ to [CH₂(SO₃)₂]²⁻ has been achieved. The reaction mixture was cooled down to 20° C. The precipitated K₂[CH₂(SO₃)₂] was isolated by suction filtration and washed three times with 100 ml of water. The product was dried 6 hours at 80° C. under atmospheric pressure.

Yield: 175 g (86.7%)

Composition:

| | | |
|---|---|---|
| Assay: | 99.7% | (Titration) |
| Br—: | 111 ppm | (Ion-chromatography) |
| SO₃²⁻: | <50 ppm | (Ion-chromatography) |
| SO₄²⁻: | 54 ppm | (Ion-chromatography) |

Purification by Recrystallization

175 g of K₂[CH₂(SO₃)₂] were suspended in 600 ml of water. The suspension was heated to about 100° C. At a pressure of about 40 mbar, 362 ml of water were evaporated out of the mixture. The remaining suspension was cooled down to 20° C. The precipitated K₂[CH₂(SO₃)₂] was isolated by suction filtration and dried 6 h at 80° C. under atmospheric pressure.

Yield: 165.4 g (94.5%)

Overall yield: 81.9%

Composition:

| | | |
|---|---|---|
| Assay: | 99.8% | (Titration) |
| Br—: | <10 ppm | (potentiometric titration) |
| SO₃²⁻: | <50 ppm | (Ion-chromatography) |
| SO₄²⁻: | <50 ppm | (Ion-chromatography) |

### EXAMPLE 2

Preparation of K₂[CH₂(SO₃)₂]

A mixture of 774 g K₂SO₃-solution (45% in water), 6 g of [(C₄H₉)₄N]Br and 0.8 g KI in 200 ml of water, was heated to 70° C. Within a period of 16 hours, 140 g of CH₂Br₂ were transferred under the surface of the reaction mixture. Afterwards the reaction mixture was heated up to 90° C. After a total reaction time of 24 hours, K₂[CH₂(SO₃)₂] started to precipitate. After 24 hours at 90° C., complete conversion of CH₂Br₂ to [CH₂(SO₃)₂]²⁻ has been achieved. The reaction mixture was cooled to 20° C. The precipitated K₂[CH₂(SO₃)₂] was isolated by suction filtration and washed three times with 100 ml of water. The product was dried 6 hours at 80° C. under atmospheric pressure.

Yield: 173 g (85.7%)

Composition:

| | | |
|---|---|---|
| Assay: | 99.4% | (Titration) |
| Br—: | 130 ppm | (Ion-chromatography) |
| SO₃²⁻: | <50 ppm | (Ion-chromatography) |
| SO₄²⁻: | 95 ppm | (Ion-chromatography) |

### EXAMPLE 3

Preparation of CH₂(SO₃H)₂

173.05 g Na₂SO₃, 7.5 g [(C₄H₉)₄N]Br, 1 g KI were dissolved in 750 ml of water. The mixture was heated to 80° C. Within a period of 16 hours, 173.8 g of CH₂Br₂ have been added under the surface of the stirred reaction mixture. After completion of the addition of CH₂Br₂, 104.54 g of Na₂SO₃ were added to the reaction mixture. Afterwards the mixture was heated to 90° C. for 72 hours. Than 50 ml of HCl-solution (37%) were added to the solution within a period of 2 hours. Afterwards the mixture was diluted with a solution of 75 g of BaCl₂×2H₂O in 500 ml of water and cooled down to about 20° C. After 2 hours, the precipitated BaSO₄ was separated by filtration. The filtrate was passed over a ion exchange column (cation exchanger type: Permutit RSP 100). Afterwards water was distilled out of the solution at a pressure of app. 50 mbar within a temperature range of 40 to 130° C. The remaining high viscous liquid was diluted with water to generate a 50% solution of CH₂(SO₃H)₂.

Yield: 298.5 g (84.7%)

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A process for producing an alkyldisulfonic acid alkali metal salt which comprises heating an aqueous solution of about two molar equivalents of an alkali metal sulfite to a temperature of from about 60° C. to about 90° C.; adding about one molar equivalent of a dihaloalkane to the aqueous alkali metal sulfite solution at a temperature of about 60° C. to about 90° C.; and then separating the resulting alkyldisulfonic acid alkali metal salt from the reaction solution.

2. Process of claim 1 wherein the dihaloalkane comprises dibromomethane, diiodomethane, bromochloromethane, bromoiodomethane or combinations thereof.

3. The process of claim 1 wherein the alkali metal sulfite comprises potassium sulfite.

4. The process of claim 1 wherein the alkali metal sulfite comprises sodium sulfite.

5. The process of claim 1 wherein the dihaloalkane is added at a pressure of about 1.1 atmospheres or less.

6. The process of claim 1 wherein the dihaloalkane is added at a pressure of about 1.01 atmospheres or less.

7. The process of claim 1 wherein the dihaloalkane is added at about atmospheric pressure.

8. The process of claim 1 wherein the aqueous alkali metal sulfite solution further comprises a catalyst selected from the group consisting of an alkali metal iodide, a tetraalkyl ammonium salt, and combinations thereof.

9. The process of claim 1 wherein the aqueous alkali metal sulfite solution further comprises a catalyst selected from the group consisting of potassium iodide, sodium iodide, lithium iodide, tetrabutylammonium bromide, methyltributylammonium bromide, methyltributylammonium chloride, and combinations thereof.

10. The process of claim 1 wherein the resulting alkyldisulfonic acid alkali metal salt comprises about 50 ppm or less of bromides.

11. The process of claim 1 wherein the resulting alkyldisulfonic acid alkali metal salt comprises about 100 ppm or less of sulfates.

12. The process of claim 1 wherein the yield of alkyldisulfonic acid alkali metal salt is about 80% or more based on the amount of amount of dibromomethane.

13. A process for producing methanedisulfonic acid potassium salt which comprises heating an aqueous solution of about two molar equivalents of potassium sulfite to a temperature of from about 60° C. to about 90° C.; adding about one molar equivalent of dibromomethane to the aqueous potassium sulfite solution at a temperature of from about 60° C. to about 90° C.; and then separating the resulting methanedisulfonic acid potassium salt from the reaction solution.

14. The process of claim 13 which is conducted at a pressure of about 1.1 atmospheres or less.

15. The process of claim 13 which is conducted at about atmospheric pressure.

16. The process of claim 13 wherein the aqueous potassium sulfite solution further comprises a catalyst selected from the group consisting of potassium iodide, a tetraalkyl ammonium salt, and combinations thereof.

17. The process of claim 13 wherein the aqueous alkali metal sulfite solution further comprises a catalyst selected from the group consisting of potassium iodide, sodium iodide, lithium iodide, tetrabutylammonium bromide, methyltributylammonium bromide, methyltributylammonium chloride, and combinations thereof.

18. The process of claim 13 wherein the resulting methanedisulfonic acid potassium salt comprises about 50 ppm or less of bromide.

19. The process of claim 13 wherein the yield of methanedisulfonic acid potassium salt is about 80% or more based on the amount of amount of dibromomethane.

20. A process for producing an alkyldisulfonic acid which comprises heating an aqueous solution of about two molar equivalents of an alkali metal sulfite to a temperature of from about 60° C. to about 90° C.; adding about one molar equivalent of a dihaloalkane to the aqueous alkali metal sulfite solution at a temperature of about 60° C. to about 90° C.; separating the resulting alkyldisulfonic acid alkali metal salt from the reaction solution; and then reacting the alkyldisulfonic acid alkali metal salt with an ion exchange polymer.
